(19) United States
(12) Patent Application Publication
(10) Pub. No.: US 2012/0258440 A1
(43) Pub. Date: Oct. 11, 2012

(54) METHOD OF DECIDING DETACHMENT TIME FOR CELL, METHOD OF SUBCULTURING CELL AND APPARATUS FOR SUBCULTURING CELL USING THE SAME

(75) Inventors: Neon Cheol Jung, Gyeonggi-do (KR); Keunchang Cho, Seoul (KR)

(73) Assignee: LOGOS BIOSYSTEMS, INC., Gyeonggi-do (KR)

(21) Appl. No.: 13/440,104

(22) Filed: Apr. 5, 2012

(30) Foreign Application Priority Data
Apr. 5, 2011 (KR) 10-2011-0031273

(51) Int. Cl. G01N 21/75; C12M 1/40; C12Q 3/00

(52) U.S. Cl. 435/3; 435/29; 435/288.7

(57) ABSTRACT

A method of deciding detachment time of cells, and a method and apparatus for subculturing the cells using the method. The method of deciding detachment time of the cells from a culturing vessel using a separation enzyme in subculturing the cells includes: a photographing step of photographing an image of the cells treated with the enzyme; a computing step of calculating a contrast value of the photographed image, and computing a change rate of the contrast value; a comparison step of repeating the photographing step and the computing step until an increasing change rate of the contrast value is reduced to a predetermined change rate or less; and a reporting step of reporting a time to remove the enzyme from the cells and to detach the cells from the culturing vessel when the increasing change rate of the contrast value is reduced to the predetermined change rate or less.

### TECHNICAL FIELD

The present invention relates to a method of deciding a detachment time of cells, and a method and an apparatus for subculturing the cells by using the method. More particularly, the present invention relates to a method of easily deciding a detachment time of cells so that the cells' damage can be prevented, and a method and an apparatus for subculturing the cells by using the method, wherein the damage may be caused when a separation enzyme used in detachment of the cells from a culturing vessel is treated for greater than a predetermined time.

### BACKGROUND ART

In general, attached cells are cultured while being adhered on a culturing vessel. When cells fill the culturing vessel by being proliferated, the proliferated cells are required to be transferred to a new culturing vessel through dilution. This is called subculturing.

For subculturing, a separation enzyme (such as trypsin) for detaching cells from the culturing vessel is treated. Most separation enzymes are protease, and thus continuously cause damage to the cells after an appropriate time period. Then, this has a negative effect on the growth of the cells, and in a serious case, the cells are subject to death.

Accordingly, in the subculturing of cells, it is very important to treat the separation enzyme at an appropriate concentration, for an appropriate time period. For this, researchers generally observe the shapes of cells by a microscope while continuously monitoring the extent of detachment of the cells from the culturing vessel.

However, this process is a subjective process which depends on naked eye determination of a researcher. Thus, it is not easy to decide an appropriate time to detach the cells from the culturing vessel. Furthermore, such decision is in actuality impossible in an unmanned culturing environment where researchers cannot continuously make observations through a microscope.

### DISCLOSURE

### Technical Problem

Therefore, the present invention has been made in view of the above-mentioned problems, and the present invention provides a method of easily deciding a detachment time of cells so that the cells' damage can be prevented, wherein the damage may be caused when a separation enzyme used in detachment of the cells from a culturing vessel is treated for greater than a predetermined time.

Also, the present invention provides a method and an apparatus of easily subculturing cells by using such a method of deciding the detachment time of the cells, in an unmanned culturing environment, for example, even in a case of using an automatic cell culturing apparatus.

Meanwhile, the technical problem of the present invention is not limited to that mentioned above. A person skilled in the art can clearly understand that there exist other technical problems that have not been mentioned.

### Technical Solution

In accordance with an aspect of the present invention, there is provided a method of deciding a detachment time of cells when the cells are detached from a culturing vessel by using a separation enzyme in subculturing of the cells, the method including: a photographing step of photographing an image of the cells treated with the separation enzyme; a computing step of calculating a contrast value of the photographed image, and computing a change rate of the contrast value; a comparison step of repeating the photographing step and the computing step until an increasing change rate of the contrast value is reduced to a predetermined change rate or less; and a reporting step of reporting a time to remove the separation enzyme from the cells and to detach the cells from the culturing vessel when the increasing change rate of the contrast value is reduced to the predetermined change rate or less.

In the method, the computing step includes the step of computing the change rate of the contrast value from the calculated contrast value and a preset contrast value.

Herein, when the photographing step and the computing step are repeated through the comparison step, the preset contrast value includes a contrast value that has been calculated in the computing step before the contrast value used in computation of the change rate of the contrast value is calculated.

Especially, when the photographing step and the computing step are repeated n (n is a natural number≧2) times through the comparison step, the preset contrast value includes a contrast value that has been calculated in an n−1^{th} computing step just before an n^{th} repetition.

Also, the computing step includes the step of computing the change rate of the contrast value from a gradient with respect to time on a virtual graph on which contrast values calculated by repeating the photographing step and the computing step through the comparison step are connected by time when the calculated contrast values are arranged by time.

The inventive method may further include an error preventing step of preventing the reporting step from wrongly reporting the time to remove the separation enzyme from the cells and to detach the cells from the culturing vessel when the change rate of the contrast value is not increased or is equal to or less than a predetermined change rate in the comparison step right after treatment of the cells with the separation enzyme.

Herein, the error preventing step includes the step of continuously repeating the photographing step and the computing step until a time during which the photographing step and the computing step are repeated is equal to or longer than a preset first time period, or includes the step of continuously repeating the photographing step and the computing step until the contrast value calculated in the computing step is equal to or larger than a preset threshold value.

Herein, the preset first time period includes a time period having a start point at which the cells are treated with the separation enzyme and an end point at which the change rate of the contrast value starts to be continuously increased for a certain time.

Also, the preset threshold value includes the contrast value calculated at a point of time when the change rate of the contrast value starts to be continuously increased for a certain time.

Meanwhile, the inventive method may further include an infinite loop preventing step of reporting that the photographing step and the computing step are continuously repeated in an infinite loop, after a preset second time period elapse, in order to prevent the photographing step and the computing step from being continuously repeated in the infinite loop.

Herein, the preset second time period includes a time period having a start point at which the cells are treated with the separation enzyme and an end point corresponding to a time point after the preset first time period elapse.

According to another aspect of the present invention, there is provided a method of deciding a detachment time of cells, the method including: a first step of obtaining a plurality of images of the cells by time by sequentially photographing the cells in culturing; a second step of calculating a contrast value from each of the images of the cells; and a third step of determining a time to detach the cells from a culturing vessel by using the contrast value.

Herein, the third step includes the step of computing a change rate of the contrast value from the contrast value and deciding a time, at which the change rate of the contrast value is reduced to a preset change rate or less, as the time to detach the cells.

Herein, the change rate of the contrast value is computed by a gradient with respect to time on a virtual graph on which contrast values calculated in the second step are connected by time.

Also, the change rate of the contrast value is computed by a gradient obtained from a first contrast value corresponding to any one of the contrast values calculated in the second step and a preset contrast value.

Herein, the preset contrast value includes a second contrast value corresponding to any other contrast value of the contrast values calculated in the second step prior to a time when the first contrast value used to obtain the gradient is calculated.

Especially, the second contrast value includes a contrast value calculated from an n−1^{th} photographed image of the cells when the first contrast value includes a contrast value calculated from an n^{th} (n is a natural number≧2) photographed image of the cells.

According to a further aspect of the present invention, there is provided a method of subculturing cells, the method including the steps of: adhering the cells to a culturing vessel; proliferating the cells; treating the proliferated cells with a separation enzyme; deciding a detachment time of the proliferated cells by using the above described method of deciding a detachment time of cells; removing the separation enzyme treated in the cells right after detachment time of the proliferated cells is decided; and detaching the cells from the culturing vessel.

According to a still further aspect of the present invention, there is provided a subculturing apparatus including: a culturing vessel for holding the cells treated with a separation enzyme; an objective lens for enlarging an image of the cells treated with the separation enzyme, which is disposed at an upper side or a lower side of the culturing vessel; an imaging device for photographing the image enlarged by the objective lens such that the photographing step in the method of deciding the detachment time of the cells is carried out; a treatment device adapted to interwork with the imaging device such that the computing step and the comparison step in the method of deciding the detachment time of the cells are carried out; and an alarming device for carrying out the reporting step in the method of deciding the detachment time of the cells.

Herein, when the photographing step and the computing step in the method of deciding the detachment time of the cells are repeated n (n is a natural number≧2) times by the treatment device, the objective lens adjusts a focus by using the contrast value calculated in an n−1^{th} computing step just before an n^{th} repetition, thereby allowing a clear image to be photographed even when the image of the cells treated with the separation enzyme is repeatedly photographed by the imaging device.

Especially, a position of the focus is set as a position at a predetermined distance from the cells treated with the separation enzyme when the focus is adjusted using the contrast value calculated in the n−1^{th} computing step just before the n^{th} repetition.

Meanwhile, the alarming device includes a communication unit capable of communicating with a portable terminal such that a time to remove the separation enzyme treated in the cells and to detach the cells from the culturing vessel is selectively identified in the portable terminal.

Moreover, the inventive apparatus may further include a light source device for adjusting a brightness of an image photographed by the imaging device, which is disposed in a direction facing the objective lens with respect to the culturing vessel.

### Advantageous Effects

In the inventive method of deciding a detachment time of cells, a time to detach the cells by using a contrast value on the cells' image is reported so that the cells' damage can be prevented, wherein the damage may be caused when a separation enzyme used in detachment of the cells from a culturing vessel is treated for greater than a predetermined time.

Also, in the method and the apparatus for subculturing cells by using the inventive method of deciding the detachment time of the cells, a time to detach the cells is decided without a researcher's determination by the naked eye. Thus, even in an unmanned culturing environment, for example, in a case of using an automatic cell culturing apparatus, it is possible to subculture the cells.

### DESCRIPTION OF THE DRAWINGS

The foregoing and other objects, features and advantages of the present invention will become more apparent from the following detailed description when taken in conjunction with the accompanying drawings in which:

FIG. 1 is a flow chart showing a method of subculturing cells, according to an embodiment of the present invention;

FIG. 2 is a flow chart showing a method of deciding a detachment time of cells, according to an embodiment of the present invention;

FIG. 3 is an image of cells, photographed right after treatment of the cells with a separation enzyme;

FIG. 4 is an image of cells, photographed at a detachment time of the cells from a culturing vessel;

FIG. 5 is a graph on which contrast values repeatedly calculated in the computing step in a method of deciding the detachment time of cells according to an embodiment of the present invention are arranged by time; and

FIG. 6 is a schematic view illustrating a part of a subculturing apparatus according to an embodiment of the present invention.

### MODE FOR INVENTION

Hereinafter, exemplary embodiments of the present invention will be described with reference to the accompanying drawings. However, well-known functions and constructions are omitted to provide a clear and concise description of the present invention.

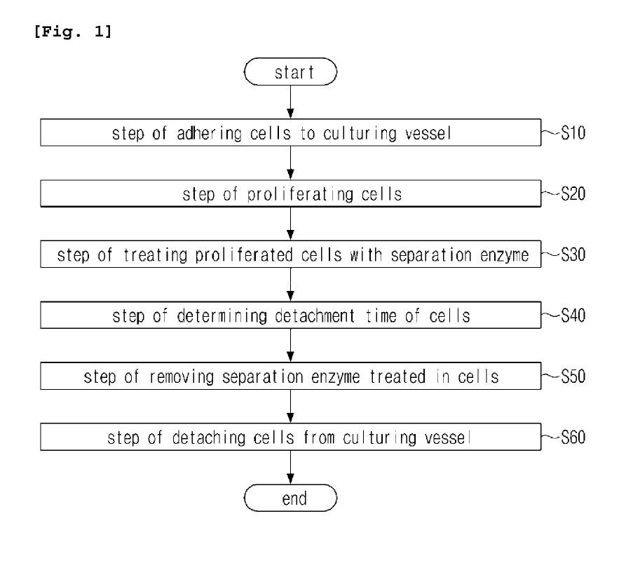

First, referring to FIGS. 1 to 5, a method of subculturing cells, according to an embodiment of the present invention, and a method of deciding a detachment time of the cell, according to an embodiment of the present invention, which is used in the subculturing method will be described.

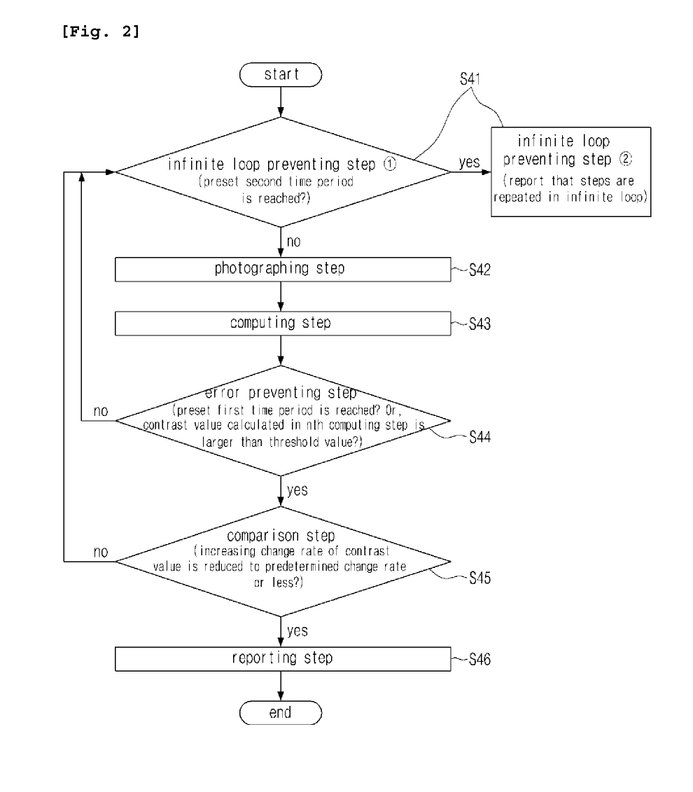

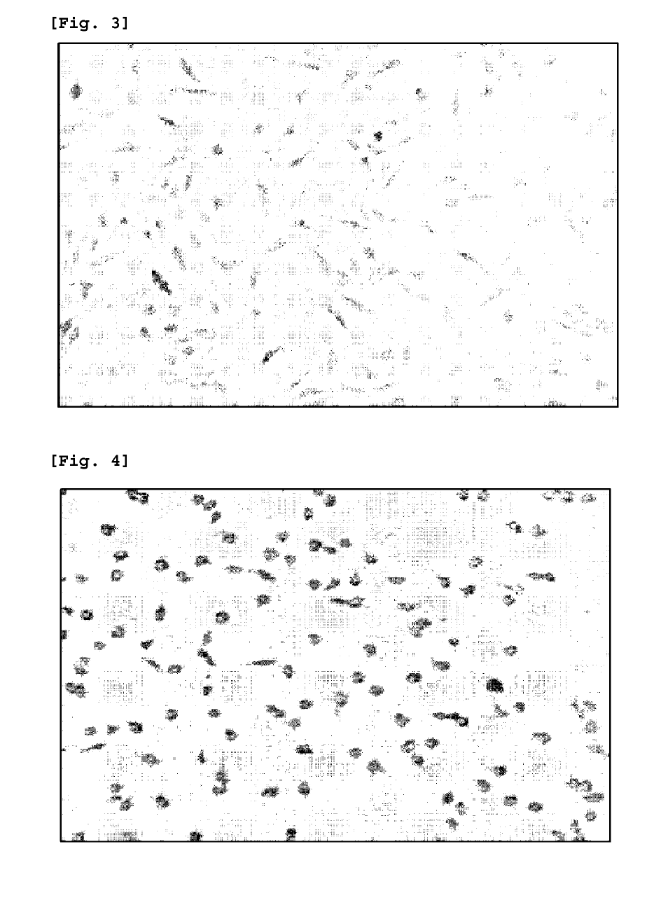

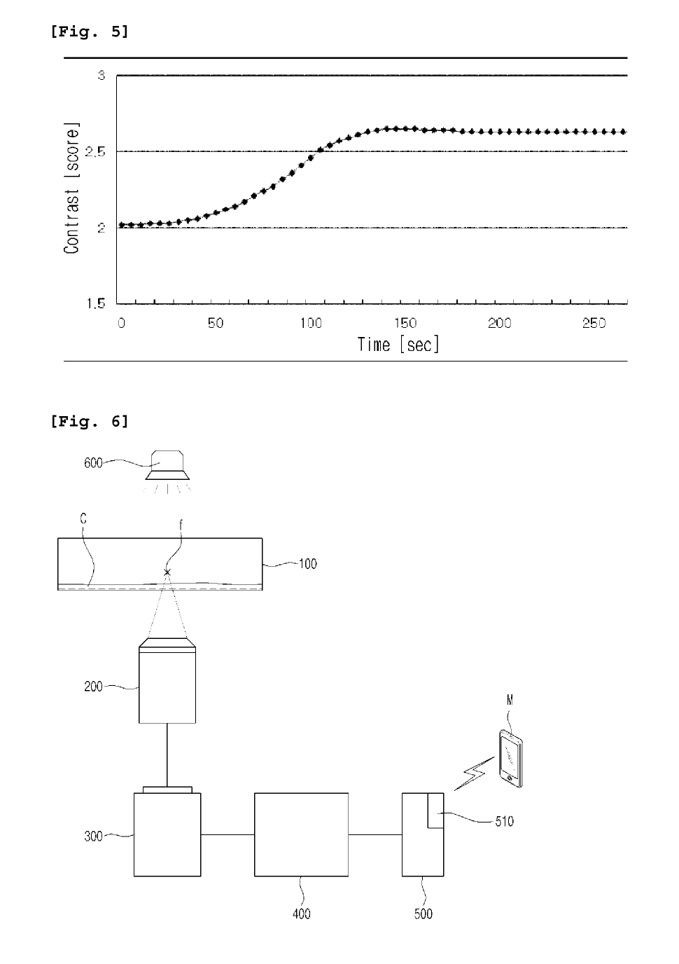

Herein, FIG. 1 is a flow chart showing a method of subculturing cells, according to an embodiment of the present invention; FIG. 2 is a flow chart showing a method of deciding a detachment time of cells, according to an embodiment of the present invention; FIG. 3 is an image of cells, photographed right after treatment of the cells with a separation enzyme; FIG. 4 is an image of cells, photographed at a detachment time of the cells from a culturing vessel; and FIG. 5 is a graph on which contrast values repeatedly calculated in the computing step in a method of deciding the detachment time of cells according to an embodiment of the present invention are arranged by time.

As shown in FIG. 1, according to an embodiment of the present invention, a method of subculturing cells includes the steps of: adhering cells to a culturing vessel (S10); proliferating the cells (S20); treating the proliferated cells with a separation enzyme (S30); deciding a detachment time of the cells (S40); removing the separation enzyme treated in the cells (S50); and detaching the cells from the culturing vessel (S60).

In the step S10 of adhering cells to a culturing vessel, some of the cells proliferated in an existing culturing vessel are transferred and settled to a new culturing vessel. Herein, it is obvious to a person skilled in the art that the existing culturing vessel and the new culturing vessel have the same physical and chemical conditions in the subculturing.

Meanwhile, in the step S20 of proliferating the cells, the number of individuals of the cells is increased through the cells' own cell division process. This step is naturally performed with a lapse of time as long as the environmental condition allowing the cells to be divided is satisfied.

After the step S10 of adhering the cells to the culturing vessel, and the step S20 of proliferating the cells are carried out, some of the proliferated cells are transferred to the new culturing vessel. For this, the following steps are carried out.

In other words, once the cells are proliferated up to certain extent, the cells are detached from the culturing vessel by using the separation enzyme such as trypsin. Then, the step S10 of adhering the cells to the culturing vessel, and the step S20 of proliferating the cells are prepared to be repeatedly carried out.

In order to detach the proliferated cells from the culturing vessel, although omitted in FIG. 1, a step of removing medium adhered to the proliferated cells in the existing culturing vessel, and a step of washing the cells with PBS or BSS in order to remove medium remaining on a cell surface, the medium inhibiting the activity of the separation enzyme such as trypsin, are carried out.

Then, the step S30 of treating the proliferated cells with the separation enzyme such as trypsin is carried out. Herein, as described in the background art, since most separation enzymes are protease, it is required to use a separation enzyme having an appropriate concentration according to the kind of the cells.

After the step S30 of treating the proliferated cells with the separation enzyme such as trypsin is carried out, the step S40 of deciding a detachment time of the cells by using the inventive method of deciding the detachment time of the cells is carried out.

Herein, the inventive method of deciding the detachment time of the cells, as shown in FIG. 2, includes an infinite loop preventing step S41, a photographing step S42, a computing step S43, an error preventing step S44, a comparison step S45, and a reporting step S46.

The infinite loop preventing step S41 is to prevent the photographing step S42 and the computing step S43 from being continuously repeated in an infinite loop. In other words, the infinite loop preventing step S41 is to prevent the photographing step S42 and the computing step S43 from being continuously repeated instead of being stopped by the error preventing step S44, or the comparison step S45 when cells do not exist in the culturing vessel, or are observed only in a very small area.

For this, after a preset second time period elapse, the infinite loop preventing step S41 notifies a user or one component of a subculturing apparatus (which will be described later) that the photographing step S42 and the computing step S43 are continuously repeated.

Herein, if the preset second time period is set to be shorter than a preset first time period, the photographing step S42, the computing step S43, and the comparison step S45 may be not carried out any more. Accordingly, the start point of the preset second time period may be set as a time when the cells are treated with the separation enzyme, and the end point may be set as a time after the preset first time period elapse.

As described above, when it is notified that the photographing step S42 and the computing step S43 are continuously repeated, the user or one component of the subculturing apparatus may perform a step of determining again if cells exist in the culturing vessel, or a step of enlarging an observed area.

When the infinite loop preventing step S41 is not carried out, the photographing step S42 and the computing step S43 are sequentially carried out. Herein, the photographing step S42 is to take an image of the cells treated with the separation enzyme, and the computing step S43 is to calculate a contrast value of a photographed image, and to compute a change rate of the calculated contrast value.

Herein, in the computing step S43, it is not required to compute a contrast value in the whole of the image photographed in the photographing step S42. In other words, only a part of the photographed image may be selected to compute the contrast value of the photographed image. This reduces the calculation time.

Also, the contrast value calculated in the computing step S43 generally indicates a difference value in brightness between highlights and shadows in an image, and may be calculated by various methods. More specifically, as an example, the value may be calculated by the following equation [mathematical equation 1] or [mathematical equation 2].

$$\begin{matrix}contrast = \frac{{\sum }_{x = 0}^{W - 1} {\sum }_{y = 0}^{L} \vert I ( x , y ) - I ( x + 1 , y ) \vert}{W \times L} & [ mathematical equation 1 ] \\ contrast = \frac{{\sum }_{x = 0}^{W - 1} {\sum }_{y = 0}^{L} {\{ I ( x , y ) - I ( x + 1 , y ) \}}^{2}}{W \times L} & [ mathematical equation 2 ]\end{matrix}$$

Herein, W represents the number of width pixels of an image used in the contrast calculation, and L represents the number of height pixels of an image used in the contrast calculation. Also, I(x,y) represents brightness of each coordinate value when a culturing vessel is plotted on a two dimensional coordinate (x-y coordinate).

As described above, after the photographing step S42 and the computing step S43 are carried out, the comparison step S45 of determining if the cells are detached from the culturing vessel by using the contrast value calculated in the computing step S43 may be carried out.

Specifically, the comparison step S45 is to repeatedly carry out the photographing step S42 and the computing step S43 until an increasing change rate of a contrast value is decreased to a predetermined change rate or less.

This comparison step S45 is based on that the contrast value is increased while cells are detached from the culturing vessel by a separation enzyme, but after most of the cells are detached from the culturing vessel, the contrast value is not increased any more but unchanged or rather decreased.

In other words, right after the cells are treated with the separation enzyme, as shown in FIG. 3, a cell image having a low contrast value is photographed. However, with a lapse of time, as the separation enzyme is activated, the cells are detached from the culturing vessel. Then, when the cells are required to be detached from the culturing vessel, as shown in FIG. 4, a cell image having a high contrast value is photographed. However, images having increasing contrast values are not continuously photographed. In other words, as shown in FIG. 5, at the point of time after 150 [sec], when most of the cells are detached from the culturing vessel, the contrast value of a photographed image may be equal to or slightly smaller than that in the previous time period.

In the comparison step S45 using such a characteristic, it is determined that at the point of time when the contrast value calculated in the computing step S43 is not increased any more, but unchanged or rather decreased, that is, at the point of time when an increasing change rate of a contrast value is reduced to a predetermined change rate or less, most of the cells are detached from the culturing vessel.

Herein, there are various methods for calculating an increasing change rate of a contrast value, used as a reference in the comparison step S45. However, specifically, the following example will be described.

Herein, in the following description with reference to the graph of FIG. 5, a predetermined change rate will be divided into a positive number and a negative number. However, naturally, this may be set by a user in consideration of the kind of cells, and the culturing method/period.

As one example, a change rate of a contrast value may be computed by using a calculated contrast value and a preset contrast value. Herein, the preset contrast value may be a value fixed as a contrast value calculated from an image photographed by a user at the point of time when most of the cells are detached from the culturing vessel.

However, the preset contrast value may be not limited to the fixed value. While the photographing step S42 and the computing step S43 are repeated n times (n is a natural number≧2) through the comparison step S45, the preset contrast value may be a contrast value calculated in the n−1^{th} computing step just before the n^{th} repetition.

For example, in the comparison step S45, on the graph shown in FIG. 5, at the point of 70 [sec], 2.17, that is, a contrast value calculated in the 13^{th} repeated computing step, is compared to 2.21, that is, a contrast value calculated in the 14^{th} repeated computing step. Herein, the contrast value calculated in the 14^{th} repeated computing step is larger than the contrast value calculated in the 13^{th} repeated computing step. The change rate of the contrast value is a positive number, and is computed to be increased. Thus, the comparison step S45 commands a 15^{th} repetition of the photographing step S42 and the computing step S43. Herein, it is obvious to a person skilled in the art that the infinite loop preventing step S41 is repeated again.

However, in the comparison step S45, on the graph shown in FIG. 5, at the point of 150 [sec], 2.65, that is, a contrast value calculated in the 29^{th} repeated computing step, is compared to 2.64, that is, a contrast value calculated in the 30^{th} repeated computing step. Herein, the contrast value calculated in the 30^{th} repeated computing step is smaller than the contrast value calculated in the 29^{th} repeated computing step. The change rate of the contrast value is a negative number, and is a predetermined change rate or less. Thus, the comparison step S45 commands the photographing step S42 and the computing step S43 to be not repeated any more.

Meanwhile, the preset contrast value may be other values than the contrast value calculated in the n−1^{th} computing step. In other words, in some cases, by using a contrast value calculated in the n−2^{nd} or n−3^{rd} computing step, the increasing change rate of a contrast value may be computed. It is natural that a contrast value is calculated in the n−2^{nd} or n−3^{rd} computing step when the contrast value calculated in the n−1^{th} computing step is not used.

As described above, before the comparison step S45 is carried out, from a gradient obtained from a specific contrast value, a change rate of a contrast value may be determined. However, the present invention is not limited thereto. Contrast values which are calculated by repeating the photographing step S42 and the computing step S43 may be arranged by time, and a virtual graph of the contrast values arranged with respect to time may be generated. In other words, as shown in FIG. 5, a two dimensional graph having an x-axis with respect to time, and a y-axis with respect to contrast values may be generated so that on the graph, from a gradient with respect to a specific time, a change rate of a contrast value can be computed.

In other words, as described above, there are various methods for determining a change rate of a contrast value so as to determine if an increasing change rate of a contrast value is reduced to a predetermined change rate or less. The gist of the present invention is that a detachment time of cells is decided from a variously obtained change rate.

Meanwhile, as described above, the comparison step S45 may be carried out right after the photographing step S42 and the computing step S43. Further, the error preventing step S44 may be carried out right after the photographing step S42 and the computing step S43 so as to previously prevent the reporting step S46 from wrongly reporting the detachment time of the cells from the culturing vessel.

Right after the cells are treated with the separation enzyme, a change rate of a contrast value is insignificant. Thus, although the cells are not detached from the culturing vessel, it may be determined that a contrast value calculated in the n^{th} computing step is equal to or smaller than a contrast value calculated in the n−1^{th} computing step, in the comparison step S45. Thus, it may be wrongly determined that the cells are completely detached.

In other words, the error preventing step S44 is to prevent an error that may occur at the point of time before about 50 [sec] on the graph shown in FIG. 5.

There is no limitation in time or method of the error preventing step S44 as long as it is possible to prevent the error. More specifically, hereinafter, two methods will be exemplified.

First, the error preventing step S44 is to continuously repeat the photographing step S42 and the computing step S43 until a time during which the photographing step S42 and the computing step S43 are repeated is equal to or longer than the preset first time period.

Herein, the preset first time period may have a start point at which the cells are treated with the separation enzyme, and an end point at which a contrast value calculated in the n^{th} computing step of the comparison step S45 starts to become continuously greater than a contrast value calculated in the n−1^{th} computing step for a certain time.

In other words, since the user previously knows that a change rate of a contrast value is insignificant until the point of time before about 50 [sec] on the graph shown in FIG. 5, this interval is set as a first time period. Then, the photographing step S42 and the computing step S43 are continuously repeated until 50 [sec], so as to prevent the above described error from occurring.

Second, the error preventing step S44 is to continuously repeat the photographing step S42 and the computing step S43 until a contrast value calculated in the computing step S43 is equal to or greater than a preset threshold value.

Herein, the preset threshold value may be a contrast value of the cells' image photographed at the point of time when a contrast value calculated in the n^{th} computing step starts to become continuously greater than a contrast value calculated in the n−1^{th} computing step for a certain time in the comparison step S45.

In other words, from about 50 [sec] on the graph shown in FIG. 5, a change rate of a contrast value is significantly increased. Herein, the contrast value at this point of time, that is, 2.10, is set as a threshold value. Then, in the computing step S43, until the contrast value (2.10) is calculated, the photographing step S42 and the computing step S43 are continuously repeated so as to prevent the above described error from occurring.

Meanwhile, after the error preventing step S44 and the comparison step S45, when a contrast value calculated in the comparison step S45 is equal to or smaller than the preset contrast value, the reporting step S46 of reporting the time to remove the separation enzyme treated in the cells, and to detach the cells from the culturing vessel is finally carried out.

In other words, the reporting step S46 is to report the time to detach the cells from the culturing vessel to the user or one component of the subculturing apparatus.

After the step S40 of deciding the detachment time of the cells in the inventive method of subculturing the cells is carried out as specifically described above, the step S50 of removing the separation enzyme treated in the cells, and the step S60 of detaching the cells from the culturing vessel are carried out.

Herein, there is no need to sequentially carry out the step S50 of removing the separation enzyme treated in the cells, and the step S60 of detaching the cells from the culturing vessel. In other words, any one step of them may be firstly carried out. However, when the subculturing apparatus is automatized, the steps may be carried out in accordance with the algorithm determining the automatization order.

Meanwhile, in the removal of the separation enzyme, various methods may be used. In general, a chemical having an ingredient capable of neutralizing the separation enzyme is treated to remove the separation enzyme.

For example, in a case where trypsin is used as a separation enzyme, the step S60 of detaching the cells from the culturing vessel may be firstly carried out, and then the step S50 of removing the separation enzyme treated in the cells by placing the detached cells in medium containing trypsin neutralizing serum may be carried out.

Next, referring to FIG. 6, according to an embodiment of the present invention, the configuration and the operation of a subculturing apparatus will be described.

Herein, FIG. 6 shows a schematic view showing a part of the subculturing apparatus according to an embodiment of the present invention.

As shown in FIG. 6, the subculturing apparatus according to an embodiment of the present invention may include a culturing vessel 100, an objective lens 200, an imaging device 300, a treatment device 400 and an alarming device 500.

Herein, the culturing vessel 100 is a component for holding cells treated with a separation enzyme. The culturing vessel 100 may be detachably combined with the subculturing apparatus according to an embodiment of the present invention. Also, the culturing vessel 100 may be made of a homogenous transparent material so that the cells can be easily observed, and an enlarged image of the cells can be well obtained through the objective lens 200.

Meanwhile, the objective lens 200 is a component for enlarging an image of the cells, which is disposed at the upper side or lower side of the culturing vessel 100. The objective lens 200 may be integratedly configured with the imaging device 300, or separately exchangeably configured for convenience of magnification adjustment.

In the objective lens 200 as configured above, the focus may be configured to be automatically adjusted in such a manner that it is possible to prevent deviation of the focus when an image of the cells is firstly photographed by the imaging device 300 or when an image of the cells treated with the separation enzyme is repeatedly photographed.

In other words, when an image of the cells is initially photographed, a stage interworking with the objective lens 200 and an image contrast value obtained at each height are used to adjust the focus. By this, the method of deciding the detachment time of the cells can be operated without an error even in an unmanned culturing environment.

Also, while the cells treated with the separation enzyme are detached from the culturing vessel 100, their shape is gradually changed. Thus, in a case where the focus of the objective lens 200 is fixed, the focus of the objective lens 200 may be deviated from the cells while the image of the cells is repeatedly photographed. Accordingly, when the photographing step S42 and the computing step S43 are repeated n times by the treatment device 400 in the method of deciding the detachment time of the cells, the focus of the objective lens 200 may be adjusted by using a contrast value calculated in the n−1^{th} computing step just before the n^{th} repetition. Thus, by this process, although the image of the cells is repeatedly photographed, it is possible to photograph a clear image.

Herein, when the focus of the objective lens 200 is adjusted by using the calculated contrast value, it is advantageous that the focus position (f) is set as a position at a predetermined distance from the cells (C) treated with the separation enzyme. This is because when the cells adhered to the bottom of the culturing vessel 100 are detached, they are generally slightly raised in a round shape from the bottom of the culturing vessel 100.

Meanwhile, the imaging device 300 is a component for carrying out the photographing step S42 in the above described inventive method of deciding the detachment time of the cells. In other words, the imaging device 300 photographs an image enlarged through the objective lens 200, and transfers the photographed image to the treatment device 400.

Meanwhile, the treatment device 400 is a component for carrying out the computing step S43 and the comparison step S45 in the above described inventive method of deciding the detachment time of the cells. In other words, the treatment device 400 interworks with the imaging device 300, while calculating a contrast value from a cells' image obtained from the imaging device 300, and comparing the calculated contrast value to a preset contrast value so as to determine the detachment time of the cells. It is not necessary to attach the treatment device 400 to the inventive subculturing apparatus. The treatment device 400 may be a peripheral computer that can communicate with the subculturing apparatus.

Meanwhile, the alarming device 500 is a component for carrying out the reporting step S46 in the above described inventive method of deciding the detachment time of the cells. In other words, the alarming device 500 notifies the user of the time to remove the separation enzyme treated in the cells and to detach the cells from the culturing vessel 100.

As described above, by the alarming device 500, the user can easily grasp the time to remove the separation enzyme treated in the cells, and carry out the above described step S50 of removing the separation enzyme treated in the cells, and the above described step S60 of detaching the cells from the culturing vessel.

Herein, in the case that the user operates another work at an area distant from the inventive subculturing apparatus, the alarming device 500 may have a communication unit 510 that can communicate with a portable terminal (M) possessed by the user. In other words, through the communication unit 510, the user can more easily determine the time to selectively detach the cells from the culturing vessel 100.

Meanwhile, the inventive subculturing apparatus including these components may automatically perform the entire process for subculturing the cells by another component (that is not described).

Herein, the above described alarming device 500 may be included in another device (not shown) for notifying of each step of the entire process of subculturing cells. Also, the alarming device 500 may be operated in such a manner that it directly provide a treatment signal to a device (not shown) for detaching the cells from the culturing vessel 100 in the inventive subculturing apparatus without notifying the user of the time to remove the separation enzyme treated in the cells.

Meanwhile, the inventive subculturing apparatus may further include the light source device 600. The light source device 600 is a component for adjusting the brightness of an image photographed by the imaging device 300, which may be disposed in a direction facing the objective lens 200 with respect to the culturing vessel 100.

Herein, when light is directly input to the objective lens 200 from the light source device 600, the light may be directly input to the imaging device 300 optically connected to the objective lens 200. Thus, it may be difficult to adjust the brightness of the image. For this case, a condenser lens (not shown) between the light source device 600 and the objective lens 200, or a reflective mirror (not shown) for adjusting a light amount between the objective lens 200 and the imaging device 300 may be additionally provided.

### INDUSTRIAL APPLICABILITY

Although several exemplary embodiments of the present invention have been described for illustrative purposes, those skilled in the art will appreciate that various modifications, additions and substitutions are possible, without departing from the scope and spirit of the invention as disclosed in the accompanying claims.

1. A method of deciding a detachment time of cells when the cells are detached from a culturing vessel by using a separation enzyme in subculturing of the cells, the method comprising:
a photographing step of photographing an image of the cells treated with the separation enzyme;
a computing step of calculating a contrast value of the photographed image, and computing a change rate of the contrast value;
a comparison step of repeating the photographing step and the computing step until an increasing change rate of the contrast value is reduced to a predetermined change rate or less; and
a reporting step of reporting a time to remove the separation enzyme from the cells and to detach the cells from the culturing vessel when the increasing change rate of the contrast value is reduced to the predetermined change rate or less.

2. The method as claimed in claim 1, wherein the computing step comprises the step of computing the change rate of the contrast value from the calculated contrast value and a preset contrast value.

3. The method as claimed in claim 2, wherein when the photographing step and the computing step are repeated through the comparison step, the preset contrast value comprises a contrast value that has been calculated in the computing step before calculating the contrast value used in computation of the change rate of the contrast value is calculated.

4. The method as claimed in claim 3, wherein, when the photographing step and the computing step are repeated n (n is a natural number 2) times through the comparison step, the preset contrast value comprises a contrast value that has been calculated in an n−1^{th} computing step just before an n^{th} repetition.

5. The method as claimed in claim 1, wherein the computing step comprises the step of computing the change rate of the contrast value from a gradient with respect to time on a virtual graph on which contrast values calculated by repeating the photographing step and the computing step through the comparison step are connected by time when the calculated contrast values are arranged by time.

6. The method as claimed in claim 1, further comprising an error preventing step of preventing the reporting step from wrongly reporting the time to remove the separation enzyme from the cells and to detach the cells from the culturing vessel when the change rate of the contrast value is not increased or is equal to or less than a predetermined change rate in the comparison step right after treatment of the cells with the separation enzyme.

7. The method as claimed in claim 6, wherein the error preventing step comprises the step of continuously repeating the photographing step and the computing step until a time during which the photographing step and the computing step are repeated is equal to or longer than a preset first time period, or comprises the step of continuously repeating the photographing step and the computing step until the contrast value calculated in the computing step is equal to or larger than a preset threshold value.

8. The method as claimed in claim 7, wherein the preset first time period comprises a time period having a start point at which the cells are treated with the separation enzyme and an end point at which the change rate of the contrast value starts to be continuously increased for a certain time.

9. The method as claimed in claim 7, wherein the preset threshold value comprises the contrast value calculated at a point of time when the change rate of the contrast value starts to be continuously increased for a certain time.

10. The method as claimed in claim 7, further comprising an infinite loop preventing step of reporting that the photographing step and the computing step are continuously repeated in an infinite loop, after a preset second time period elapses, in order to prevent the photographing step and the computing step from being continuously repeated in the infinite loop.

11. The method as claimed in claim 10, wherein the preset second time period comprises a time period having a start point at which the cells are treated with the separation enzyme and an end point corresponding to a time point after the preset first time period elapse.

12. A method of deciding a detachment time of cells, the method comprising:
a first step of obtaining a plurality of images of the cells by time by sequentially photographing the cells in culturing;
a second step of calculating a contrast value from each of the images of the cells; and
a third step of determining a time to detach the cells from a culturing vessel by using the contrast value.

13. The method as claimed in claim 12, wherein the third step comprises the step of computing a change rate of the contrast value from the contrast value and deciding a time, at which the change rate of the contrast value is reduced to a preset change rate or less, as the time to detach the cells.

14. The method as claimed in claim 13, wherein the change rate of the contrast value is computed by a gradient with respect to time on a virtual graph on which contrast values calculated in the second step are connected by time.

15. The method as claimed in claim 13, wherein the change rate of the contrast value is computed by a gradient obtained from a first contrast value corresponding to any one of the contrast values calculated in the second step and a preset contrast value.

16. The method as claimed in claim 15, wherein the preset contrast value comprises a second contrast value corresponding to any other contrast value of the contrast values calculated in the second step prior to a time when the first contrast value used to obtain the gradient is calculated.

17. The method as claimed in claim 16, wherein the second contrast value comprises a contrast value calculated from an n−1^{th} photographed image of the cells when the first contrast value comprises a contrast value calculated from an n^{th} (n is a natural number≧2) photographed image of the cells.

18. A method of subculturing cells, the method comprising the steps of:
adhering the cells to a culturing vessel;
proliferating the cells;
treating the proliferated cells with a separation enzyme;
deciding a detachment time of the proliferated cells by using the method of deciding a detachment time of cells, as claimed in claim 1;
removing the separation enzyme treated in the cells right after detachment time of the proliferated cells is decided; and
detaching the cells from the culturing vessel.

19. A subculturing apparatus using the method of deciding a detachment time of cells, as claimed in claim 1, the subculturing apparatus comprising:
a culturing vessel for holding the cells treated with a separation enzyme;
an objective lens for enlarging an image of the cells treated with the separation enzyme, which is disposed at an upper side or a lower side of the culturing vessel;
an imaging device for photographing the image enlarged by the objective lens such that the photographing step in the method of deciding the detachment time of the cells is carried out;
a treatment device adapted to interwork with the imaging device such that the computing step and the comparison step in the method of deciding the detachment time of the cells are carried out; and
an alarming device for carrying out the reporting step in the method of deciding the detachment time of the cells.

20. The subculturing apparatus as claimed in claim 19, wherein, when the photographing step and the computing step in the method of deciding the detachment time of the cells are repeated n (n is a natural number≧2) times by the treatment device, the objective lens adjusts a focus by using the contrast value calculated in an n−1^{th} computing step just before an n^{th} repetition, thereby allowing a clear image to be photographed even when the image of the cells treated with the separation enzyme is repeatedly photographed by the imaging device.

21. The subculturing apparatus as claimed in claim 20, wherein a position of the focus is set as a position at a predetermined distance from the cells treated with the separation enzyme when the focus is adjusted using the contrast value calculated in the n−1^{th} computing step just before the n^{th} repetition.

22. The subculturing apparatus as claimed in claim 19, wherein the alarming device comprises a communication unit capable of communicating with a portable terminal such that a time to remove the separation enzyme treated in the cells and to detach the cells from the culturing vessel is selectively identified in the portable terminal.

23. The subculturing apparatus as claimed in claim 19, further comprising a light source device for adjusting a brightness of an image photographed by the imaging device, which is disposed in a direction facing the objective lens with respect to the culturing vessel.
